(19) United States
(12) Patent Application Publication
(10) Pub. No.: US 2005/0054918 A1
(43) Pub. Date: Mar. 10, 2005

(54) Method and system for treatment of atrial fibrillation and other cardiac arrhythmias

(76) Inventor: Jasbir S. Sra, Pewaukee, WI (US)

Correspondence Address: JANSSON, SHUPE & MUNGER, LTD, 245 MAIN STREET, RACINE, WI, 53403, US

(21) Appl. No.: 10/932,487

(22) Filed: Sep. 2, 2004

Related U.S. Application Data
(60) Provisional application No. 60/500,106, filed on Sep. 4, 2003.

(51) Int. Cl. A61B 5/05

(52) U.S. Cl. 600/427; 604/21

(57) ABSTRACT

A method is provided for treatment of a heart arrhythmia such as atrial fibrillation that includes obtaining cardiac image data using a digital imaging system, generating a 3D model of a cardiac chamber and surrounding structures from such cardiac image data, registering the 3D model with an interventional system, visualizing this registered 3D model on the interventional system, positioning a catheter apparatus within the cardiac chamber, visualizing the catheter apparatus over the registered 3D model of the cardiac chamber upon the interventional system, navigating the catheter apparatus within the cardiac chamber utilizing this registered 3D model, and delivering biological material through the catheter apparatus to heart tissue at select locations within the cardiac chamber. Preferably, the biological material are transplanted cells or antibodies. In another aspect of the invention, a system for treatment of heart arrhythmias is provided that has a digital imaging system to obtain cardiac image data, an image generation system to generate a 3D model of a cardiac chamber and its surrounding structures from this cardiac image data, a workstation to register the 3D model onto an interventional system so that the registered 3D model can be visualized upon the interventional system, and a catheter apparatus to deliver biological material such as transplanted cells or antibodies to heart tissue within this cardiac chamber at certain select locations, the catheter apparatus being visualized upon the interventional system over the registered 3D model.

### RELATED APPLICATION

This application claims the benefit of U.S. Provisional Application No. 60/500,106 filed on Sep. 4, 2004.

### FIELD OF THE INVENTION

This invention relates generally to methods and systems for treatment of atrial fibrillation and other cardiac arrhythmias and, in particular, to methods and systems for delivering biological material to a chamber inside the heart.

### BACKGROUND OF THE INVENTION

Atrial fibrillation is an arrhythmia of the heart in which the atria or upper chambers of the heart stop contracting as they fibrillate. Premature atrial contraction (extra beats) originating in the pulmonary veins can act as triggers and initiate paroxysms of atrial fibrillation. The inability to reproducibly induce premature beats and precisely identify the ostium or junction of the pulmonary veins with the left atrium due to the complex three-dimensional geometry of the left atrium makes prohibitive the use of ablation therapy in many patients. There is also a risk of complications such as stroke, bleeding around the heart and narrowing of the pulmonary veins during radio-frequency catheter ablation procedures.

Studies have found activity that is suggestive of the presence of conduction tissue at the left atrial-pulmonary vein junction. Thus, a new approach directed at blocking conduction at a cellular or molecular level by delivering biological material that would block conduction across cells could provide significant advantages in the treatment of this complex arrhythmia. Such delivery systems could include the transplantation of cells or the injection of antibodies.

This approach could also be beneficial to treating other arrhythmias and other conditions if precise localization and delivery of cells, antibodies and similar biological substances including genes were possible.

### SUMMARY OF THE INVENTION

One aspect of this invention provides a method for treatment of a heart arrhythmia having the steps of (1) obtaining cardiac image data using a digital imaging system, preferably a computer tomography (CT) system, (2) generating a 3D model of a cardiac chamber and surrounding structures from this cardiac image data, (3) registering the 3D model with an interventional system, (4) visualizing this registered 3D model on the interventional system, (5) positioning a catheter apparatus within the cardiac chamber, (6) visualizing the catheter apparatus over the registered 3D model of the cardiac chamber upon the interventional system, (7) navigating the catheter apparatus within the cardiac chamber utilizing this registered 3D model, and (8) delivering biological material through the catheter apparatus to heart tissue at select locations within the cardiac chamber.

In certain preferred embodiments, the biological material being delivered by the catheter apparatus are transplanted cells that can alter electrical impulses at these select locations within the heart. Highly preferred is where the transplanted cells are myoblasts.

Another desirable embodiment is where the biological material delivered to heart tissue within the cardiac chamber are antibodies such that electrical impulses at the selected locations are altered by these antibodies.

It is most desirable that the interventional system be a fluoroscopic system. More desirable is where the heart arrhythmia is atrial fibrillation and the 3D model is of the left atrium and pulmonary veins. Highly desirable embodiments find the catheter apparatus having a main body with a central lumen that is adapted to deliver biological material and a control mechanism coupled to the main body such that the delivery of the biological material from the main body is controlled.

In another aspect of this invention, a system is provided for treatment of a heart arrhythmia that has a digital imaging system to obtain cardiac image data, an image generation system to generate a 3D model of a cardiac chamber and its surrounding structures from this cardiac image data, a workstation to register the 3D model onto an interventional system so that the registered 3D model can be visualized upon the interventional system, and a catheter apparatus to deliver biological material to heart tissue within this cardiac chamber at certain select locations, the catheter apparatus being visualized upon the interventional system over the registered 3D model.

Desirable cases of this system find the biological material delivered to be transplanted cells, most preferably myoblasts. Also highly desirable is where the biological material are antibodies.

Preferred embodiments of this system are where the interventional system is a fluoroscopic system. Most preferred embodiments find the digital imaging system to be a computer tomography (CT) system. In certain preferred cases, the heart arrhythmia is atrial fibrillation and the 3D model is of the left atrium and pulmonary veins. Highly preferred is where the catheter apparatus includes a main body having a central lumen adapted to the delivery of the biological material and a control mechanism coupled to the main body to control such delivery from the apparatus.

### BRIEF DESCRIPTION OF THE DRAWINGS

FIG. 1 is a schematic overview of a system for treatment of a heart arrhythmia in accordance with this invention with an enlarged longitudinal cross-section of a portion of the catheter.

FIG. 2A depicts 3D cardiac images of the left atrium.

FIG. 2B illustrates localization of a standard mapping and ablation catheter over an endocardial view of the left atrium registered upon an interventional system.

FIG. 3 is a flow diagram of a method for treatment of atrial fibrillation and other cardiac arrhythmias in accordance with this invention.

### DETAILED DESCRIPTION OF PREFERRED EMBODIMENTS

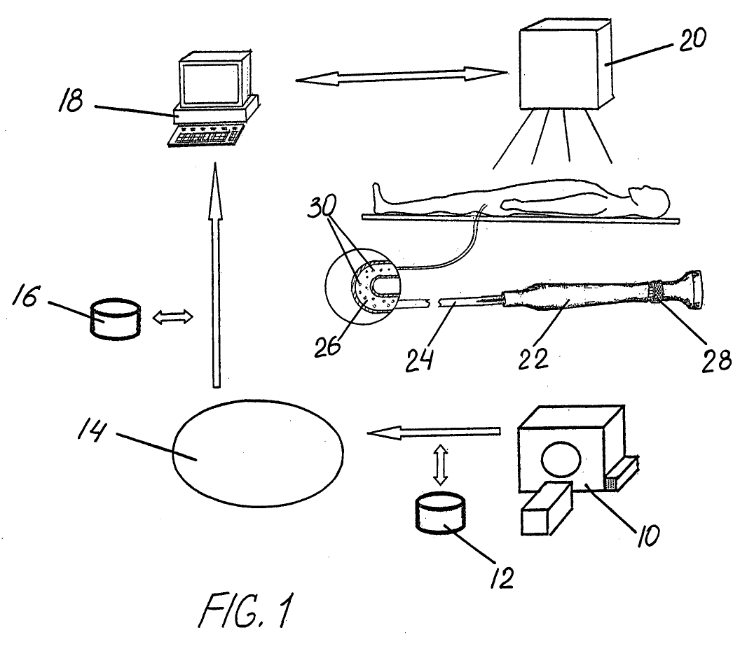

FIG. 1 illustrates a schematic overview of an exemplary system for the treatment of a heart arrhythmia such as atrial fibrillation in accordance with this invention. A digital imaging system such as a CT scanning system 10 is used to acquire image data of the heart. Although the embodiments discussed hereinafter are described in the context of a CT scanning system, it will be appreciated that other imaging systems known in the art, such as MRI and ultrasound, are also contemplated.

Cardiac image data 12 is a volume of consecutive images of the heart collected by CT scanning system 10 in a continuous sequence over a short acquisition time. The shorter scanning time through use of a faster CT scanning system and synchronization of the CT scanner with the QRS on the patient's ECG signal reduces the motion artifacts in images of a beating organ like the heart. The resulting cardiac image data 12 allows for reconstruction of images of the heart that are true geometric depictions of its structures.

Cardiac image data 12 is then segmented using protocols optimized for the left atrium and pulmonary arteries by image generation system 14. It will be appreciated that other chambers of the heart and their surrounding structures can be acquired in a similar manner. Image generation system 14 further processes the segmented data to create a 3D model 16 of the left atrium and pulmonary arteries using 3D surface and/or volume rendering. Additional post-processing can be performed to create navigator (view from inside) views of these structures.

3D model 16 is then exported to workstation 18 for registration with an interventional system such as a fluoroscopic system 20. The transfer of 3D model 16, including navigator views, can occur in several formats such as the DICOM format and geometric wire mesh model. Information from CT scanning system 10 will thus be integrated with fluoroscopic system 20. Once 3D model 16 is registered with fluoroscopic system 20, 3D model 16 and any navigator views can be seen on the fluoroscopic system 20.

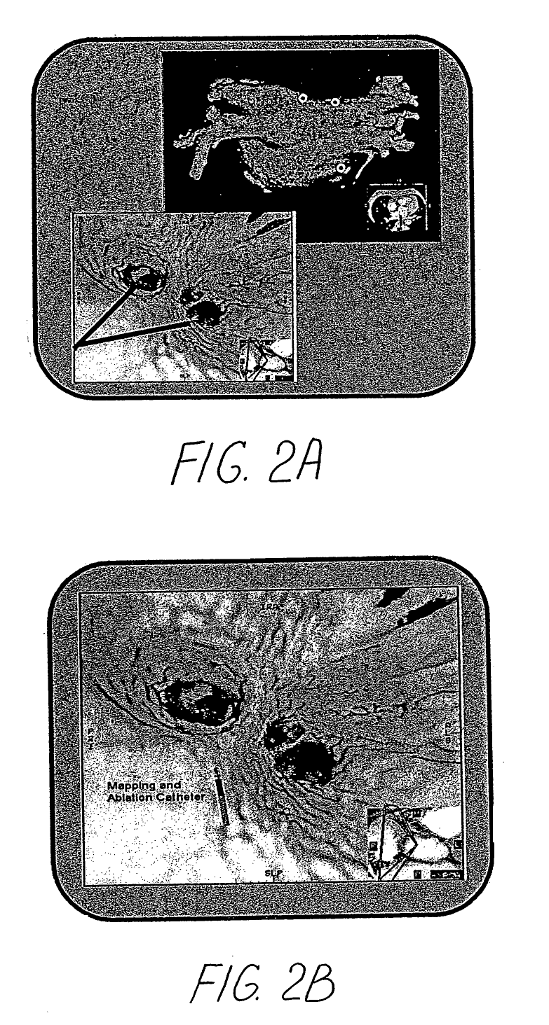

A detailed 3D model of the left atrium and the pulmonary veins, including endocardial or inside views, is seen in FIG. 2A. The distance and orientation of the pulmonary veins and other strategic areas can be calculated in advance from this 3D image to create a roadmap for use during the ablation procedure.

Using a transeptal catheterization, which is a standard technique for gaining access to the left atrium, a catheter apparatus 22, having a flexible catheter 24 with a central lumen 26, is introduced into the left atrium. Catheter 24 is visualized on the fluoroscopic system 20 over the registered 3D model 16. Catheter 24 can then be navigated in real-time over 3D model 16 to the appropriate site within the left atrium. FIG. 2B illustrates localization of a standard mapping and ablation catheter over an endocardial view of the left atrium registered upon an interventional system.

Catheter apparatus 22 is provided with a control mechanism 28 for opening and closing the distal end of lumen 26. Upon filling lumen 26 with biological material 30, catheter apparatus 22 can be used as a delivery device for the release of biological material 30 at specifically selected locations within the heart. After catheter 24 has been guided to a site identified as a strategic area whose electrical conductivity needs to be altered or blocked, control mechanism 28 is actuated to deliver biological material 30 such as transplanted cells at that site. Such transplanted cells could be myoblastic or smooth muscle cells. Antibodies can also be injected in this manner to alter or block abnormal electrical activity at the cellular level, especially in responding to antigens that may be responsible for the triggering of impulses that initiate atrial fibrillation.

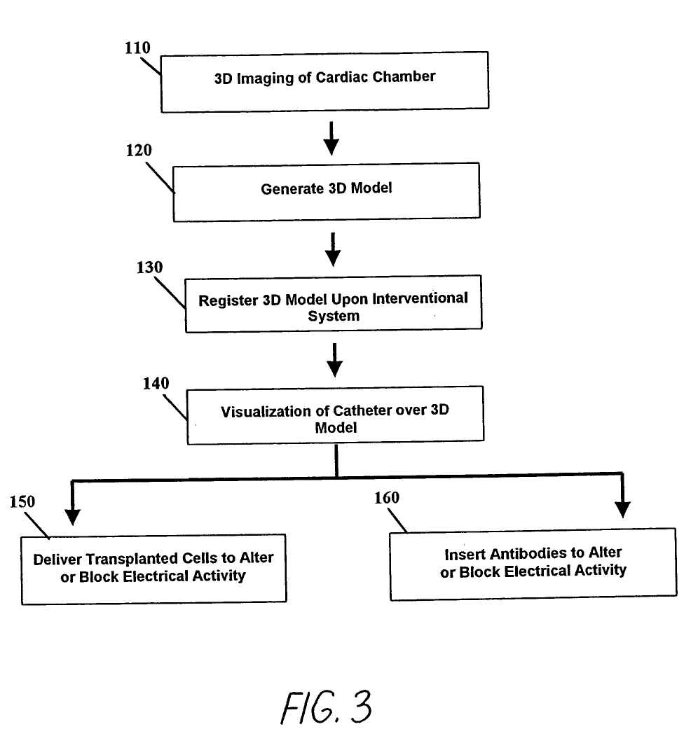

There is shown in FIG. 3 an overview of a method for ablation of atrial fibrillation and other cardiac arrhythmias in accordance with this invention. As seen in step 110, a 3D image of the heart is acquired. 3D images of the heart can be created using CT scan or MRI. At step 120, a 3D model of the chamber of interest such as the left atrium is created through segmentation of the image data using protocols optimized for the appropriate structures. Once this 3D model has been obtained, it can be stored as an electronic data file using various means of storage. The stored model can then later be transferred to a computer workstation linked to an interventional system.

As illustrated in step 130, after it has been transferred to the workstation, the 3D model is registered with the interventional system. The registration process allows medical personnel to correlate this 3D model of the cardiac chamber with the interventional system that is being used with a particular patient so that it can be visualized during the interventional procedure.

The following step 140 involves visualization of a catheter that has been positioned within the left atrium over the registered 3D model. This permits the catheter to be navigated inside the chamber in real-time over this registered image to the locations selected for the treatment to be performed.

In step 150, transplanted cells such as myoblasts are released from a central lumen of the catheter at the selected site to alter or block electrical activity across that location. Alternatively, at step 160, antibodies or genes can be inserted at the site in treatment of the arrhythmia after being transported to the left atrium within the catheter's lumen.

It will be appreciated to one skilled in the art that other arrhythmias such as ventricular tachycardia can be targeted for treatment in this manner. Furthermore, automatic techniques may be used to perform any of the above steps.

Various alternatives and embodiments are contemplated as being within the scope of the following claims particularly pointing out and distinctly claiming the subject matter regarded as the invention.

1. A method for treatment of a heart arrhythmia comprising:
obtaining cardiac image data from a digital imaging system;
generating a 3D model of a cardiac chamber and surrounding structures from the cardiac image data;
registering the 3D model with an interventional system;
visualizing the registered 3D model upon the interventional system;
positioning a catheter apparatus within the cardiac chamber;
visualizing the catheter apparatus over the registered 3D model upon the interventional system;
navigating the catheter apparatus within the cardiac chamber utilizing the registered 3D model; and
delivering biological material through the catheter apparatus to heart tissue at select locations.

2. The method of claim 1 wherein the biological material are transplanted cells, whereby the transplanted cells alter electrical impulses at the select locations.

3. The method of claim 2 wherein the transplanted cells are myoblasts.

4. The method of claim 1 wherein the biological material are antibodies, whereby the antibodies alter electrical impulses at the select locations.

5. The method of claim 1 wherein the interventional system is a fluoroscopic system.

6. The method of claim 1 wherein the digital imaging system is a computer tomography (CT) system.

7. The method of claim 1 wherein the heart arrhythmia is atrial fibrillation and wherein the 3D model is of the left atrium and pulmonary veins.

8. The method of claim 1 wherein the catheter apparatus comprises:
a main body having a central lumen adapted to the delivery of biological material; and
a control mechanism coupled to the main body wherein delivery of the biological material from the main body is controlled.

9. A system for treatment of a heart arrhythmia comprising:
a digital imaging system for obtaining cardiac image data;
an image generation system for generating a 3D model of a cardiac chamber and surrounding structures from the cardiac image data;
a workstation for registering the 3D model with an interventional system to visualize the registered 3D model upon the interventional system; and
a catheter apparatus for delivering biological material to heart tissue within the cardiac chamber at select locations, whereby the catheter apparatus is visualized over the registered 3D model upon the interventional system.

10. The system of claim 9 wherein the biological material are transplanted cells, whereby the transplanted cells alter electrical impulses at the select locations.

11. The system of claim 10 wherein the transplanted cells are myoblasts.

12. The system of claim 9 wherein the biological material are antibodies, whereby the antibodies alter electrical impulses at the select locations.

13. The system of claim 9 wherein the interventional system is a fluoroscopic system.

14. The system of claim 9 wherein the digital imaging system is a computer tomography (CT) system.

15. The system of claim 9 wherein the heart arrhythmia is atrial fibrillation and wherein the 3D model is of the left atrium and pulmonary veins.

16. The system of claim 9 wherein the catheter apparatus comprises:
a main body having a central lumen adapted to the delivery of biological material; and
a control mechanism coupled to the main body wherein delivery of the biological material from the main body is controlled.
